(19) United States
(12) Patent Application Publication
(10) Pub. No.: US 2006/0134779 A1
(43) Pub. Date: Jun. 22, 2006

(54) Modulation of cell intrinsic strain to control cell modulus, matrix synthesis, secretion, organization, material properties and remodeling of tissue engineered constructs

(76) Inventors: Albert J. Banes, Hillsborough, NC (US); Jie Qi, Chapel Hill, NC (US)

Correspondence Address: THE WEBB LAW FIRM, P.C., 700 KOPPERS BUILDING, 436 SEVENTH AVENUE, PITTSBURGH, PA, 15219, US

(21) Appl. No.: 11/076,425

(22) Filed: Mar. 9, 2005

Related U.S. Application Data
(60) Provisional application No. 60/551,909, filed on Mar. 10, 2004.

(51) Int. Cl. A61H 1/00; C12N 5/08

(52) U.S. Cl. 435/325; 435/366; 435/404; 601/1

(57) ABSTRACT

The present invention provides methods for manipulating the intrinsic strain of cells by treating tissue engineered constructs or native tissue with compounds which affect the intrinsic strain setpoint of the cells in order to modulate matrix synthesis, secretion, organization and/or remodeling so that the tissues withstand in vivo mechanical forces and have the structural characteristics of host tissue which has been permanently altered by injury, atrophy or disease. The compounds include binding site peptides, ATP, UTP and related analogues, IL-1β, TGF-α, cytochalasin D, hyaluronic acid, nocodazole and others. Also provided are methods for applying a mechanical external strain to the tissues, as well as methods for modulating the expression of cytoskeletal genes that transcribe cytoskeletal proteins which regulate a cell's intrinsic strain setpoint.

### CROSS REFERENCE TO RELATED APPLICATIONS

The present application claims priority to U.S. Provisional Application No. 60/500,049, filed Sep. 4, 2003, and to U.S. Provisional Application No. 60/551,909, filed Mar. 10, 2004, which are incorporated by reference herein.

### BACKGROUND OF THE INVENTION

1. Field of the Invention

The present invention relates generally to methods of manipulating the intrinsic strain setpoint of cells and/or matrix in the biomedical science field of tissue engineering and, more specifically, relates to methods for manipulating intrinsic strain of tissue engineered constructs or native tissue in order to modulate extracellular matrix synthesis, secretion, organization and/or remodeling.

2. Description of Related Art

Orthopedic tissue engineering involves a combination of technologies derived from cell biology, materials science and mechanical engineering. In the United States, more than 100,000 patients per year undergo surgery to repair tendon or ligament injuries. The current “gold standard” for surgical repair is to use autologous tendon. However, one caveat is that during repair, the mechanical strength and structural characteristics of the host tissue are permanently altered. For example, during anterior cruciate ligament (ACL) reconstruction of the knee, often, with the use of patellar tendon, an initial loss of strength in the host tissue typically is observed from the time of implantation. A gradual increase in strength may occur, but usually the strength of the tissue never reaches its original magnitude.

Current research in connective tissue engineering has been focused on using natural materials as a matrix into which cells are seeded (Awad, H. A. et al., J. Biomed. Mater. Res., 51, 233, 2000; Awad, H. A. et al., Tissue Eng., 5, 267, 1999; Huang, D. et al., Ann. Biomed. Eng., 21, 289-1993; Kleiner, J. B. et al., J. Orthop. Res., 4, 466, 1986), or using acellular synthetic materials, such as Dacron® (Andrish, J. T. et al., Clin. Orthop., 183, 298, 1984), polytetrafluoroethylene (Bolton, C. W. et al., Clin. Orthop., 196, 175, 1985), polypropylene (Kennedy, J. C. et al., Am. J. Sports Med., 8, 1, 1980), or carbon fibers (Jenkins, D. H. et al., J. Bone Joint Surg. Br., 59, 53, 1977). Most of these synthetic materials, however, do not approximate the material properties of tendon or ligament, thus resulting in stress shielding in the natural tissue. Moreover, wear debris can result in an immunological response which ultimately leads to implant failure, resulting in the need for additional surgery. In other cases, degradation products can lead to acidification of the surrounding tissue, cell death or growth stasis, and implant failure. Thus, the current shortage of natural replacements for load-bearing tissue has created a demand for artificial tissues that can withstand in vivo mechanical forces.

Tissue development depends on dynamic interactions between cells and their matrix. The matrix is a fluid-filled network composed of collagens, proteoglycans and glycoproteins. Transmembrane integrin receptors mechanically couple the matrix to the cytoskeleton of a cell. Both the matrix and the cytoskeleton contribute to the mechanical properties of tissues. In turn, the mechanical properties of load-bearing tissues, such as blood vessels and ligaments, influence their functionality.

Cells require an appropriate degree of mechanical deformation to maintain a degree of intrinsic strain. It is well accepted that immobilization of limbs, bed rest and a reduction in the intrinsic strain value in a tissue leads to bone mineral loss, tissue atrophy, weakness and, in general, a reduction in anabolic activity and an increase in catabolic activity. On the other hand, physical activity results in anabolic effects, strengthening, an increase in tissue strength and an increase in the intrinsic strain in a tissue.

There exists a need, therefore, to fabricate tissue constructs and/or to modulate native tissues that are able to withstand in vivo mechanical forces and that have the structural characteristics of host tissue which has been permanently altered by injury, atrophy or disease.

### SUMMARY OF THE INVENTION

The present invention provides methods for manipulating the modulus or intrinsic strain of cells and/or their matrix, comprised of treating cells with compounds that affect the modulus or intrinsic strain setpoint in order to modulate integrin binding and/or extracellular matrix synthesis, secretion, organization and/or remodeling, material properties or attachment of the cells to the matrix via integrins or other integrin-like cell matrix attachments.

Compounds capable of such manipulation include, for example and without limitation, binding site peptides that involve entire sequences, peptide sequences from entire sequences or peptide mimetics or their active parts, such as collagens, elastins, fibronectins or laminins or their binding site peptides; decorin; biglycan; fibromodulin and lumican or their active parts; ligands, such as, without limitation, adenosine triphosphate (ATP), adenosine diphosphate (ADP), adenosine monophosphate (AMP), uridine triphosphate (UTP), uridine diphosphate (UDP) or uridine monophosphate (UMP); hyaluronic acid; cytokines, such as, without limitation, interleukin-1beta (IL-1β) or tumor necrosis factor-alpha (TNF-α); mediators, such as, without limitation, cytochalasin D or nocodazole or other compounds that affect the cytoskeleton and, hence, the intrinsic strain setpoint; or growth factors such as, without limitation, platelet-derived growth factor (PDGF), insulin-like growth factor (IGF-1 or 2), fibroblast growth factor (FGF), transforming growth factor-beta1 (TGF-β1), transforming growth factor-beta3 (TGF-β3) or others in the TGF-β family, connective tissue growth factor (CTGF) that promotes matrix expression or even mineralization, or other growth factors that affect cell migration, cell movement and compaction of the matrix, or matrix reorganization.

The present invention also provides methods for applying a mechanical external strain to tissue engineered constructs, comprised of uniaxially and/or biaxailly loading the construct by placing ARCTANGLE™ loading posts beneath a well of a culture plate and applying a vacuum to deform a flexible membrane downward so as to apply a uniaxial and/or biaxial strain along a long axis of the tissue engineered construct. Tissue engineered constructs can include, without limitation, human tendon internal fibroblast (HTIF)-populated bioartificial tendons, ligaments, menisci, cartilage, muscle, fascia and other connective tissues as bioartificial tissues (BATs™), including those populated by autologous, allogeneic cells or stem cells from adult or embryonic sources.

Compounds that are used to treat tissue engineered constructs according to the methods of the present invention can be added at the beginning, during or at the end of fabrication of the tissue engineered construct.

The present invention further provides methods for modulating the expression of cytoskeletal genes responsible for transcribing cytoskeletal proteins that regulate the intrinsic strain setpoint of cells, such as cells of native tissue in situ. Such cytoskeletal genes can include, without limitation, genes that transcribe cytoskeletal proteins, such as actin, myosin, α-actinin, vimentin, vinculin or titin, as well as genes that transcribe elastin or matrix metalloproteinases. The methods of the present invention also encompass the use of RNA silencing techniques or other gene expression-modulating techniques to reduce expression of the above-described genes or other genes which may impact the intrinsic strain setpoint of cells.

The present invention further provides methods for modulating gene expression of extracellular matrix proteins or peptides or modulating the binding of extracellular matrix proteins or peptides to integrins on the cell exterior and to cytoskeletal or other cytoskeletal-like modulating proteins on the cell interior, and for uniting the extracellular matrix (ECM) via integrins or other like attachments to the cytoskeleton to complete the ECM connection to the internal structures of a cell. The connections may be integrins but may also be other cell adhesion molecules that unite cells to cells.

### BRIEF DESCRIPTION OF THE DRAWINGS

Table 1 lists PCR conditions used for each gene;

Table 2 provides a comparison of modulus of elasticity and ultimate tensile strength results for mechanically conditioned and control specimens on Day 7;

FIG. 1 is an illustration of a Tissue Train® culture plate with a Delrin™ TroughLoader™ insert and an ARCTANGLE™ loading post. FIG. 1A shows a Delrin™ TroughLoader™ insert that is 35 mm in diameter and completely fills the space beneath a well of a Tissue Train® culture plate. The trough is 25 mm×3 mm×3 mm. The four holes are 1 mm in diameter and communicate with the reservoir beneath the culture plate so that a vacuum can draw the overlying rubber membrane into the trough creating a space into which cells and gel can be cast. Once the gel is cast, the TroughLoader™ is removed. To mechanically load the bioartifical tendons (BAT™), an ARCTANGLE™ loading post (FIG. 1B) is placed beneath the Tissue Train® well so that the linear sides correspond to the east and west poles of the anchors to which the linear gel is attached. Vacuum draws the flexible but inelastic anchors downward resulting in uniaxial strain on the BAT™. FIG. 1C shows a Tissue Train® culture plate with linear anchors in each well and two wells with a TroughLoader™ and Arctangle™ loading post;

FIG. 2 is a schematic diagram of one well of a Tissue Train® 6 well culture plate (top view) shown from above, the gel trough into which the rubber membrane is drawn by vacuum, the non-woven nylon mesh anchor bonded to the rubber in the sector portion and the anchor stem with collagen bonded thereon. On the side view, the anchor stem is shown free of the rubber bottom connected to the potted nylon anchor. Vacuum drawn through the TroughLoader™ holes pulls the rubber membrane downward to closely conform to the trough bay dimensions. Cells in a collagen gel then are added to the trough bay and the constructs are gelled at 37° C. in a CO₂ incubator. After gelation, vacuum is released and the cultures receive culture medium;

FIG. 3 shows the dimensions of a typical BAT™. FIG. 3A (top view) shows the dimensions of a typical BAT™ from the initial molding on day 0 through contraction phases on days 5, 7 and 14. The BAT™ assumes an hourglass shape (days 5 and 7) and finally a cylindrical shape (day 14). FIG. 3B (side view) shows one well of a Tissue Train® culture plate with a molded linear BAT™ immersed in culture medium. The rubber membrane faces an opposing lubricated Arctangle™-shaped loading post (rectangle with curved short ends). When a vacuum is applied to the well bottom, the rubber membrane deforms downward at east and west poles resulting in uniaxial elongation of the BAT™;

FIG. 4 is a graph showing growth curves for avian internal fibroblasts grown in 2D polystyrene culture dishes covalently bonded with Collagen I and BAT™ plated at 200K or 500K in collagen gels in Tissue Train® culture plates. Cells in 2D cultures entered log phase and passed through several division cycles, whereas cells in 3D gels plated at 200K cells/gel divided once and those plated at 500K cells/gel did not divide;

FIG. 5 is a graph showing dimensional analyses of BAT™ fabricated from 200K or 500K avian tendon internal fibroblasts per BAT™. A higher ratio of cells to gel matrix increased contraction rate.

FIG. 6 shows a BAT™. FIG. 6A depicts a 10× picture of a longitudinal cross section of a BAT™ cultured for 10 days in a Tissue Train® culture well, then harvested, fixed, sectioned and stained with hematoxylin and eosin (H&E). FIG. 6B is a higher magnification picture (40×) showing an epitenon-like surface layer that is two to three cells thick as well as longitudinally aligned tenocytes with elongate basophilic nuclei;

FIG. 7 shows a BAT™ in a Tissue Train® culture plate. FIG. 7A shows the BAT™ in a Tissue Train® culture plate on day 10 post-fabrication. FIGS. 7B and 7D show tendon internal fibroblasts linearly arranged in the collagen gel matrix. These cells have polymerized actin visualized after staining with rhodamine phalloidin for F actin and nuclei stained with DAPI. FIG. 7C shows randomly arranged cells at the BAT™ anchor region where stress shielding occurs;

FIG. 8 is a bar graph showing gene expression levels for Collagen I, III and XII, decorin, tenascin and B actin as markers which are highly expressed in tendon cells. Expression levels were similar for cells grown in 2D cultures on collagen bonded surfaces in BATs™ in collagen gels or in whole tendon. Cells in native tendon expressed slightly less tenascin and about 2.2 fold more Collagen XII than 2D and 3D counterparts (p<0.05);

FIG. 9 is a bar graph showing that cells in BATs™ which were mechanically loaded at 1 Hz, 1% elongation for 1 h/day for up to 5 days increased expression levels of collagen XII on day 3 (15%, p=0.05). Prolyl hydroxylase expression was increased 32% on day 3 and over two-fold on day 5 in loaded cultures (p<0.05);

FIG. 10 shows contraction curves of BATs™ in the absence or presence of 100 pM IL-1β (FIG. 10A), and recovery of elongated BATS™ after maximum stretch (FIG. 10B).

FIG. 11 is a bar graph showing the up-regulation of MMPs by IL-1β;

FIG. 12 is a bar graph showing gene expression of elastin and collagen regulated by IL-1β−/+10 μM cytochalasin D (CytoD) or 100 μg/ml GRGDTP;

FIG. 13 is a bar graph showing that IL-1β reduced cell modulus of monolayer HTIFs from young and adult patients; and

FIG. 14 is bar graphs showing that IL-1β down-regulated the expression of β-actin. FIG. 14A shows that, in 2D cultures, IL-1β reduced the expression of β-actin at days 1 and 3. At day 5, the protein level of β-actin almost recovered. FIG. 14B shows that, in 3D cultures, the message level of β-actin returned at day 3 but the recovery of proteins was delayed;

### DETAILED DESCRIPTION OF THE INVENTION

The present invention provides methods for manipulating the modulus or intrinsic strain setpoint in cells, such as tissue engineered constructs in vitro or native tissue in vivo and a forming tissue by modulating the cell's connections to its extracellular matrix (ECM) or by modulating the internal strain (actual or perceived), with or without the synergistic or antagonistic action of applied mechanical loading. Such modulation is regulated in the cell through the cell's connections to other cells or to its matrix by matrix attachment proteins, such as integrins, connections through cytoskeletal filaments, or by pathways which modulate the cell-matrix connections and/or cytoskeleton at the plasma membrane, at the endoplasmic reticulum and at the nucleus.

As used herein, the terms “extracellular matrix,” “matrix” and “substrate” are interchangeable.

As used herein, the term “native tissue” is any tissue that originates and/or is situated in a human body.

In particular, the present invention provides methods for treating an in vitro fabricated tissue engineered construct or an in situ native tissue with compounds which cause a release or engagement of cell attachment points to its matrix, such as peptides that compete for the attachment sites. Such peptides can include, without limitation, collagen, elastin or fibronectin-binding site peptides which contain an arginine-glycine-aspartic acid sequence (-RGD-), and laminin-binding peptides that contain a tyrosine-isoleucine-glycine-serine-arginine (-YIGSR-) sequence.

The present invention also provides a method for applying a mechanical external strain to tissue engineered constructs comprised of biaxially loading a tissue engineered construct by placing a circular Loading Post™ as a planar faced cylindrical post beneath a well of a culture plate and applying a vacuum to deform a flexible membrane downward so as to apply an equibiaxial strain to a tissue engineered construct.

The present invention further provides a method for applying a mechanical external strain to tissue engineered constructs comprised of uniaxially and biaxially mechanically loading the tissue engineered construct by placing an Arctangular™ loading post as a rectangle with curved short ends and then placing a circular Loading Post™ as planar-faced cylindrical posts beneath a well of a culture plate, and applying a vacuum to deform a flexible membrane downward so as to apply a uniaxial strain then an equibiaxial strain to a tissue engineered construct.

Compounds capable of such manipulation include, for example and without limitation, binding site peptides that involve entire sequences, peptide sequences from entire sequences or peptide mimetics or their active parts, such as collagens, elastins, fibronectins or laminins or their binding site peptides; decorin; biglycan; fibromodulin and lumican or their active parts; ligands, such as, without limitation, adenosine triphosphate (ATP), adenosine diphosphate (ADP), adenosine monophosphate (AMP), uridine triphosphate (UTP), uridine diphosphate (UDP) or uridine monophosphate (UMP), or nonmetabolyzable analogs of these or other like compounds; hyaluronic acid; cytokines, such as, without limitation, interleukin-1beta (IL-1β) or tumor necrosis factor-alpha (TNF-α); mediators, such as, without limitation, cytochalasin D or nocodazole or other compounds that affect the cytoskeleton and, hence, the intrinsic strain setpoint; or growth factors such as, without limitation, platelet-derived growth factor (PDGF), insulin-like growth factor (IGF-1 or 2), fibroblast growth factor (FGF), transforming growth factor-beta1 (TGF-β1), transforming growth factor-beta3 (TGF-β3) or others in the TGF-β family, connective tissue growth factor (CTGF) that promotes matrix expression or even mineralization, or other growth factors that affect cell migration, cell movement and compaction of the matrix, or matrix reorganization.

Tissue engineered constructs can include, without limitation, human tendon internal fibroblast (HTIF)-populated bioartificial tendons, ligaments, menisci, cartilage, muscle, fascia and other connective tissues as bioartificial tissues (BATs™), including those populated by autologous, allogeneic cells or stem cells from adult or embryonic sources.

Compounds that are used to treat tissue engineered constructs according to the methods of the present invention can be added at the beginning, during or at the end of fabrication of the tissue engineered construct.

The present invention further provides methods for modulating the expression of cytoskeletal genes responsible for transcribing cytoskeletal proteins that regulate the intrinsic strain setpoint of cells, such as cells of native tissue in situ. Such cytoskeletal genes can include, without limitation, genes that transcribe cytoskeletal proteins, such as actin, myosin, α-actinin, vimentin, vinculin or titin, as well as genes that transcribe elastin or matrix metalloproteinases. The methods of the present invention also encompass the use of RNA silencing techniques or other gene expression-modulating techniques to reduce expression of the above-described genes or other genes which may impact the intrinsic strain setpoint of cells.

The present invention further provides methods for modulating gene expression of extracellular matrix proteins or peptides or modulating the binding of extracellular matrix proteins or peptides to integrins on the cell exterior and to cytoskeletal or other cytoskeletal-like modulating proteins on the cell interior, and for uniting the extracellular matrix (ECM) via integrins or other like attachments to the cytoskeleton to complete the ECM connection to the internal structures of a cell. The connections may be integrins but may also be other cell adhesion molecules that unite cells to cells.

Other peptides or mediators used according to the methods of the present invention to modulate attachment of a cell to its matrix include proteoglycans, such as, without limitation, decorin, biglycan, fibromodulin, lumican, or peptides derived therefrom with similar composition, effect or action. Such compounds are capable of regulating the shape of the cell as well as its synthetic expression phenotype.

The present invention also includes adding matrix components to a tissue engineered construct at the beginning, during or at the end of fabrication of the tissue engineered construct in order to modulate its attachment to the matrix via integrins, transmembrane proteins that link the matrix components outside the cell to the cytoskeleton within the cell. Additionally, the degree of matrix remodeling can be regulated by treating the tissue engineered construct or native tissue with compounds that affect such remodeling. For example, inclusion of hyaluronic acid can reduce ECM remodeling.

In one embodiment of the present invention, a cell can be treated with compounds to modulate its intrinsic strain with or without mechanical loading of external strain. For example, cytokines, such as interleukin-1 beta (IL-1β) or tumor necrosis factor-alpha (TNF-α) can be given to the cell, which can act in at least two ways: (1) to modulate expression of cytoskeletal genes and synthesis of cytoskeletal proteins, such as, without limitation, actin, myosin, α-actinin, vimentin, vinculin, titin and others and hence to modulate the cell's intrinsic stiffness; and (2) to modulate gene expression of matrix metalloproteinases (MMPs), which when activated can degrade the matrix. Other mediators, such as, without limitation, cytochalasin b, cytochalasin D, nocodazole or colchicines, can be used to treat cells in order to interfere with actin or tubulin polymerization and thus to decrease the modulus of the cells and thus alter their internal strain.

In another embodiment of the invention, expression of matrix proteins or proteoglycans can be altered by treating cells with growth factors that increase matrix synthesis, secretion and organization, thus increasing the stiffness or modulus of the matrix. An example of such a growth factor is transforming growth factor-beta (TGF-β, such as transforming growth factor-beta1 (TGF-β1) or transforming growth factor-beta3 (TGF-β3); or connective tissue growth factor (CTGF). Other factors that are believed to increase matrix expression and increase matrix stiffness are, for example and without limitation, insulin-like growth factor 1 or 2; platelet-derived growth factor (PDGF-AA, AB, or BB); or bone morphogenetic proteins (BMPs), particularly BMP-2, 3, 7, 12 and 13. Addition of ascorbic acid or one of its forms (ascorbate or ascorbate-2-phosphate) also can increase matrix expression by increasing expression of CTGF and then increasing expression of transforming TGF-β.

In another embodiment of the invention, cells are treated with growth factors, such as are listed in the previous paragraph, which are believed to modulate the ability of the cells within a matrix to compact and organize the matrix so that it can better withstand physical forces applied by surrounding tissues, particularly muscles.

In still another embodiment of the present invention, RNA silencing techniques or other gene expression modulating techniques can be used to reduce expression of genes which affect the intrinsic strain setpoint of an in situ native tissue or an in vitro tissue engineered construct. The ability to specifically inhibit gene function in a variety of organisms utilizing antisense RNA or dsRNA-mediated interference (RNAi or dsRNA) is well-known in the field of molecular biology (see, for example, C. P. Hunter, 1999, Current Biology, 9:R440-442; Hamilton et al., 1999, Science, 286:950-952; and S. W. Ding, 2000, Current Opinions in Biotechnology, 11:152-156). Interfering RNA, either double-stranded interfering RNA (dsRNAi or dsRNA) or RNA-mediated interference (RNAi), typically comprises a polynucleotide sequence identical or homologous to a target gene, or fragment of a gene, linked directly, or indirectly, to a polynucleotide sequence complementary to the sequence of the target gene or fragment thereof. The dsRNAi may comprise a polynucleotide linker sequence of sufficient length to allow for the two polynucleotide sequences to fold over and hybridize to each other, although a linker sequence is not necessary. The linker sequence is designed to separate the antisense and sense strands of RNAi significantly enough to limit the effects of steric hindrance and allow for the formation of dsRNAi molecules and does not hybridize with sequences within the hybridizing portions of the dsRNAi molecule. The specificity of this gene silencing mechanism appears to be extremely high, blocking expression only of targeted genes, while leaving other genes unaffected. The terms “dsRNAi,” “RNAi” and “siRNA” are used interchangeably herein.

RNA containing a nucleotide sequence identical to a fragment of the target gene is preferred for inhibition; however, RNA sequences with insertions, deletions and point mutations relative to the target sequence can also be used for inhibition. Sequence identity may be optimized by sequence comparison and alignment algorithms known in the art (see Gribskov and Devereux, Sequence Analysis Primer, Stockton Press, 1991, and references cited therein) and then calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand(s); the promoters may be known inducible promoters, such as baculovirus. Inhibition may be targeted by specific transcription in the cells. The RNA strands may or may not be polyadenylated. The RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing and/or stabilization of the duplex strands.

Double stranded RNA molecules (dsRNA) may be introduced into cells with single stranded RNA molecules (ssRNA), which are sense or anti-sense RNA of known nucleotide sequences of genes which affect the intrinsic strain setpoint of a cell. Methods of introducing ssRNA and dsRNA molecules into cells are well known to the skilled artisan and include transcription of plasmids, vectors or genetic constructs encoding the ssRNA or dsRNA molecules according to this aspect of the invention. Electroporation, transfection, biolistics (a genetic engineering technique where particles are accelerated to deliver genetic material directly into cells), or other well-known methods of introducing nucleic acids into cells by other means also may be used to introduce the ssRNA and dsRNA molecules of this invention into cells.

Cells maintain an intrinsic setpoint for strain mediated by attachment to their matrix as well as arrangement of cytoskeletal filament proteins. In tissues, these attachments to collagens and/or proteoglycans impart to the cell a given shape with either extensive cell processes, as in many connective tissue cells such as those in tendon or ligament or bone, or few processes, as in chondrocytes at weight bearing cartilage.

Cells fabricate, organize and strengthen their matrix by a mechanism described as “structural tensioning,” i.e., a cell's application of force to its substrate without necessarily moving along the substrate. This mechanism is driven by “tractional structuring,” i.e., a cell's ability to move along matrix fibers and reorganize the matrix by aligning fibrils, squeezing out water and fundamentally compacting the matrix. Structural tensioning is one of the factors which influences the establishment of a particular structure of cells via the tension created by tractional structuring. Additionally, cells are able to maintain their own setpoint for a basal intrinsic strain level, which is determined in part by their connectivity to the matrix, their internal architecture that balances the external and internal forces acting on the cells, and their propensity to move along the matrix. Furthermore, cells respond to extrinsic tension by adjusting their shape, connections to their matrix and other cells, and their internal tension. Thus, cells develop an intrinsic strain value for a given extrinsic strain and attempt to modulate their cell-matrix contacts, pseudopod lengths, degree and types of cytoskeletal organization and modulus of elasticity based on this intrinsic strain value.

When cells are treated in a matrix or prior to seeding in a matrix, their attachment to the matrix and their tensional structuring of the matrix by tractional structuring are modulated. Modulation of attachment and tensional structuring also is achieved by adding ligands, such as, without limitation, adenosine triphosphate (ATP), adenosine diphosphate, (ADP), adenosine monophosphate (AMP), uridine triphosphate (UTP), uridine diphosphate (UDP) or uridine monophosphate (UMP) which cause a relaxation of the cells through a purinoceptor-driven pathway (P2Y or P2X).

It is believed, without being bound by the theory, that once the intrinsic strain setpoint of the cell is reset by altering the cytoskeleton profile, ratio and structure, the cells respond by making more matrix and/or matrix components, resetting their remodeling regimen, and making a more organized and robust matrix having greater mechanical strength. In particular, a cell can be modulated to direct matrix remodeling through matrix organization, degradation and/or matrix synthesis, which can result in increased matrix build-up and/or organization or reorganization, yielding a tissue engineered construct or native tissue with greater strength to endure the rigors of a native biomechanical environment. These processes can occur via manipulation of connections to the matrix externally, by manipulating the internal architecture of the cell, or by using both manipulations, either alone or in combination, simultaneously or sequentially, to affect the intrinsic strain setpoint of a cell.

Thus, treatment of cells according to the methods of the present invention results in cells which may express more matrix or more of a given matrix component, such as collagen, elastin or proteoglycan, or the matrix may become more highly cross-linked in response to a change in the intrinsic strain of the cells. Such alteration(s) results in a matrix that has a more native phenotype, is more organized, and is stronger so as to resist applied strain.

Cells that form tissue environments are present in three-dimensional matrices that are structural and functional. These matrices have their own particular anatomy, material structure, functional hierarchy and biomechanical properties. As a tissue develops, its cells fabricate their matrix in a given geometry according to developmental pathway cues. One pathway is a mechanical deformation pathway that likely includes both inside-out as well as outside-in components. An inside-out pathway may involve cell contraction in response to a ligand such as a growth factor, cytokine or hormone, while an outside-in pathway would involve matrix deformation which is transmitted to the cell via linkage to integrins, focal adhesion complexes, i.e., mechanosensory complexes, and the cytoskeleton, cell adhesion molecules, ion channels or other membrane-linked mechano-detection systems (Banes et al., Biochem & Cell Biology, 73, 349-365, 1995).

The methods of the present invention thus manipulate a cell's intrinsic strain setpoint by setting and resetting the setpoint, thereby modulating the organization/reorganization, modeling/remodeling and/or synthesis of the cell's matrix, chemically and biochemically. For example, a cell can be stimulated to set, reset, pause, alter, stop or accelerate the rate at which the cell(s) in a native tissue or a tissue engineered construct or normal healing tissue can reorganize its matrix. The matrix is comprised of collagens, proteoglycans and other external molecules. Additionally, the cell can regulate its cell-cell contacts as well as cell-matrix contacts. The role of matrix reorganization is to consolidate an existing matrix, i.e., to align, orient, compact, cross-link and strengthen the surrounding matrix. Compounds, such as, without limitation, ATP, UTP and analogs thereof, and channel blockers, such as, without limitation, suramin, verapamil, nifedipine or gadolinium, can be used singly or in combination in timed doses to regulate these responses. Second, cell migration and tractional structuring, as well as structural tractioning, can be stimulated. Both tractional structuring and structural tractioning of a matrix provides a strong and functional matrix which can withstand the biochemical rigors of the native environment as well as act as the repository for all biological signals in the matrix, such as growth factors, norepinephrine, epinephrine, or cytokines. Thus, when the matrix is in an appropriate orientation, it is able to provide the necessary conduits for proper mechanical signaling. Third, outside-in signaling can be modulated via regulating the degree to which cells connect to their matrix and hence receive and transduce mechanical signals. An example of outside-in signaling is deformation from the matrix through integrins to the cytoskeleton in order to activate membrane-bound complexes, which can be phosphorylated and activated to release a mediator to activate a transcription factor or to activate genes in the nucleus. Fourth, inside-out signaling can be modulated by regulating the ability of the cell to transmit signal information received by outside-in stimuli to inside-out signals. An example of inside-out signaling is the passage of inositol-tris phosphate through gap junctions, or the secretion of mediators, such as nitric oxide, prostaglandin E2, ATP or others. Finally, a cell's stiffness can be regulated by modulating cytoskeletal proteins with compounds such as, without limitation, phalloidin, cytochalasin D or B, colchicines, or other compounds that modulate, i.e., depolymerize or polymerize, the cytoskeleton.

The above-delineated concepts are supported by the finding that ATP and its nonmetabolizable analogues are able to retard gel matrix contraction in a culture system. Thus, ATP and similar analogues can be used in tissue engineering applications to modulate the modeling and remodeling rates as measured directly by the contraction rate of the gel matrix. Similarly, cytokines, such as IL-1β or TNF-α, can be used to modulate a cell's ability to interact with and compact its matrix. These agents reduce cytoskeletal mRNA expression of genes, such as actin, α-actinin, tubulin, titin and others, and apparently reduce the capacity of the cell to exert a force on the matrix. It is likely, therefore, that compounds like cytokines and ATP, as well as mechanical load, intersect at certain signaling pathways as the primary mechanism behind matrix remodeling.

The present invention thus allows for the culturing of cells in matrix material(s) either outside the body in vitro or within the body in situ for the purpose of engineering a tissue to replace, augment or repair a damaged native tissue or provide a missing tissue. Cells that are part of an engineered tissue or in a native tissue can thus be modulated by chemical ligands to alter their intrinsic strain environment such that the cells remodel the surrounding matrix to make it stronger and more organized. Thus, mediators, such as the cytokines IL-1β and TNF-α can be used to modulate both the matrix metalloproteinase (MMP) expression pattern in cells as well as the cytoskeleton pattern. This modulation can favorably affect the strength and arrangement of the cytoskeleton inside the cell as well as the matrix outside the cell. Other mediators, such as ATP or UTP, can be used to modulate the expression patterns further to reduce expression of the MMPs. Additionally, adding particular regimens of mechanical loading of the constructs can synergize with the effects of the mediators in common and/or intersecting pathways which further modulate the effects of the mediators and result in cells that can withstand mechanical loading. Doses of mediators and mechanical loading can be used that accentuate expression of collagens and elastins as well as particular cytoskeletal filaments. Furthermore, the alteration in cytoskeleton filament profiles can modulate cell stiffness resulting in a cell that can better resist externally applied or internally applied loads. Thus, the methods of the present invention can be used to manipulate a cell's expression patterns for both matrix, cell attachment proteins, cytoskeletal binding partners, pathway modulators and cytoskeletal proteins in order to yield cells and matrices which are stronger than nontreated counterparts and which can better withstand the rigors of their biomechanical environment.

The present invention is more particularly described in the following examples, which are intended to be illustrative only, as numerous modifications and variations therein will be apparent to those skilled in the art.

### EXAMPLE 1

### Bioartificial Tendons and Application of Mechanical Load

1. Introduction

Natural material such as fibrillar collagen can act as a scaffold allowing cells to integrate it into host tissue. This material can be formulated to approximate the host tissue's collagen type (generally type I collagen) and material properties and is minimally antigenic. Additionally, it would be advantageous to use a material seeded with native tendon cells because it is these cells that are responsible for normal tissue maintenance, remodeling and metabolism. Together, these ideas are the basis for the hypothesis that mechanically conditioned tendon internal fibroblasts, grown in a tethered, three-dimensional collagenous matrix, can mimic native tendon in appearance, genetic expression and biomechanical properties to create a bioartificial tendon using native tendon cells in a molded, Type I collagen matrix which can be subjected to a mechanical loading regimen.

2. Methods

Cell Culture

Avian tendon internal fibroblasts (ATIFs) were isolated from the flexor digitorum profundus tendons of 52-day-old White Leghorn chickens (n=3 different isolates). Chicken feet were obtained from a Purdue processing plant (Robbins, N.C.). Legs were washed with soap and cold water prior to tendon isolation. The flexor digitorum profundus tendons were removed from the middle toes after transection at the proximal portion of the metatarsal and distal portion of the tibiotarsus. Using sterile technique, tendons were dissected from their sheath and placed in a sterile dish of phosphate buffered saline (PBS) with 20 mM HEPES, pH 7.2 with 1× penicillin/streptomycin (100 units penicillin/100 μg streptomycin per ml (1× p/s)). Cells were subsequently isolated by sequential enzymatic digestion and mechanical disruption (13, 14). Cells were cultured until confluent in Dulbecco's Minimum Essential Medium-High Glucose (DMEM-H) with 10% fetal calf serum (FCS), 20 mM HEPES, pH 7.2, 100 mM ascorbate-2-phosphate and 1× p/s.

Fabrication of a Three-Dimensional Bioartificial Tendon

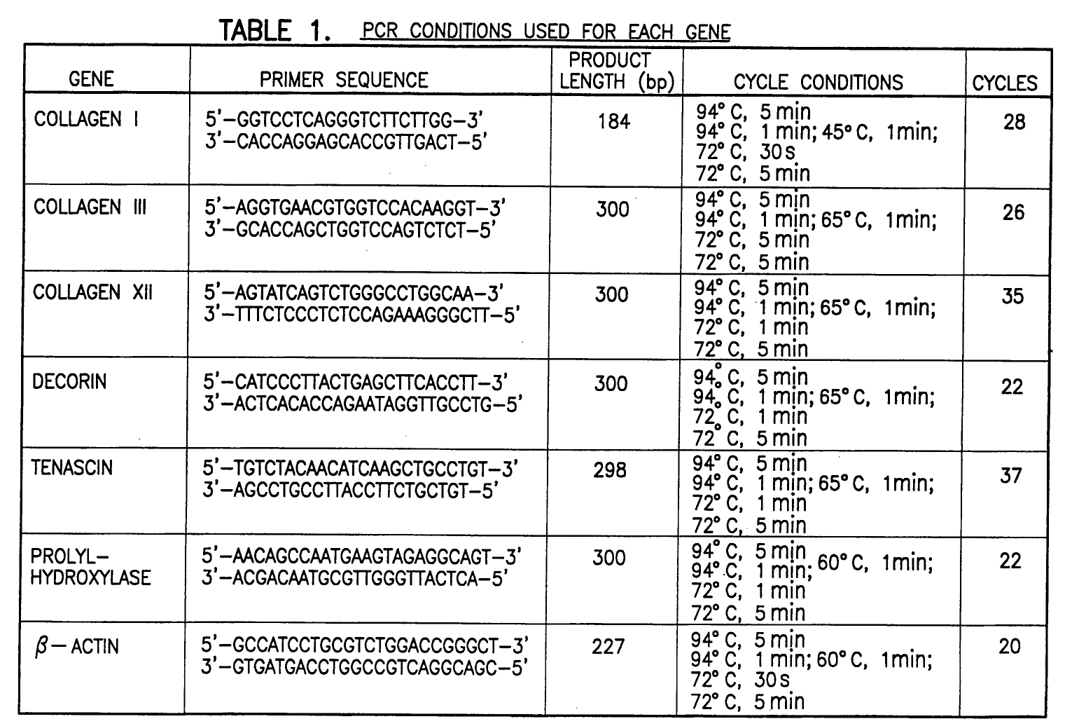

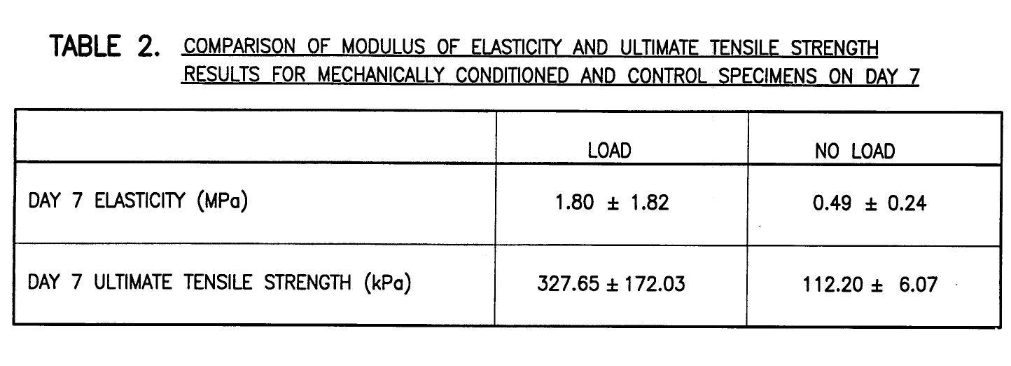

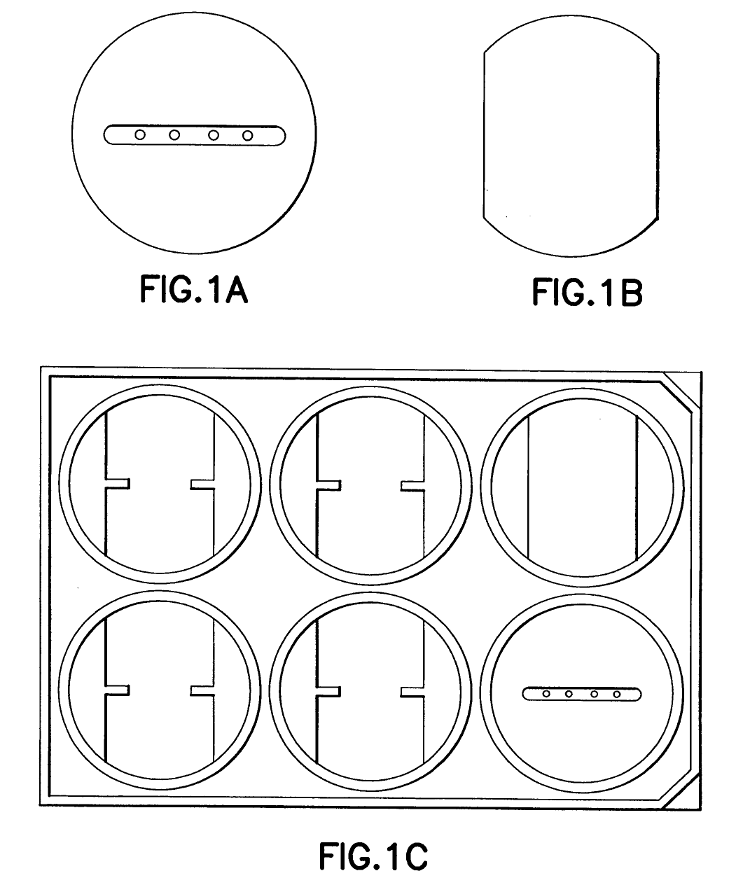

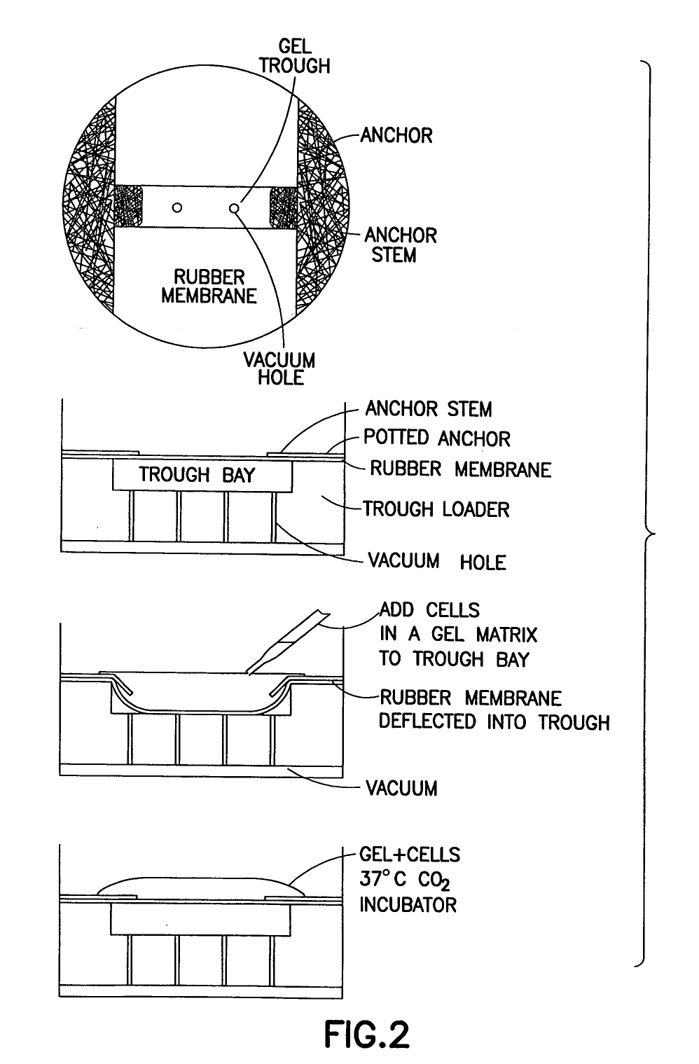

Avian tendon internal fibroblasts were enzymatically removed from a polystyrene culture plate with 0.025% trypsin. Cells were collected into a 15 ml conical tube, sedimented, washed in PBS, resuspended in 10 ml of media and counted. Collagen I (Vitrogen, Cohesion Technologies; Palo Alto, Calif.) was mixed with growth media, FBS, and neutralized to pH 7.0 with 1M sodium hydroxide. Two hundred thousand cells per 170 ul of the collagen mixture were suspended and apportioned into each well of a Tissue Train® culture plate (FIG. 1C). Linear, tethered, 3D-cell populated matrices were formed by placing the TissueTrain® culture plate atop a 4 place gasketed baseplate with planar-faced cylindrical posts inserted into centrally located, rectangular cut-outs (6 place Loading Station™ with TroughLoaders™) beneath each flexible well base, as disclosed in U.S. Pat. No. 6,472,202 and International Patent Application PCT/US01/47745, herein incorporated in their entirety by reference. (FIG. 1A). The TroughLoaders™ had vertical holes in the floor of the rectangle through which a vacuum could be applied to deform the flexible membrane into the trough. The trough provided a space for delivery of cells and matrix (FIG. 2). The baseplate was transferred into a 5% CO₂, humidified incubator at 37° C., where the construct was held in position under vacuum for 1.5 h until the cells and matrix formed a gelatinous material connected to the anchor stems. BATs™ were then covered with 3 ml per well growth medium, cultures were digitally scanned (vide infra, BAT™ contraction index) and plates were returned to the incubator.

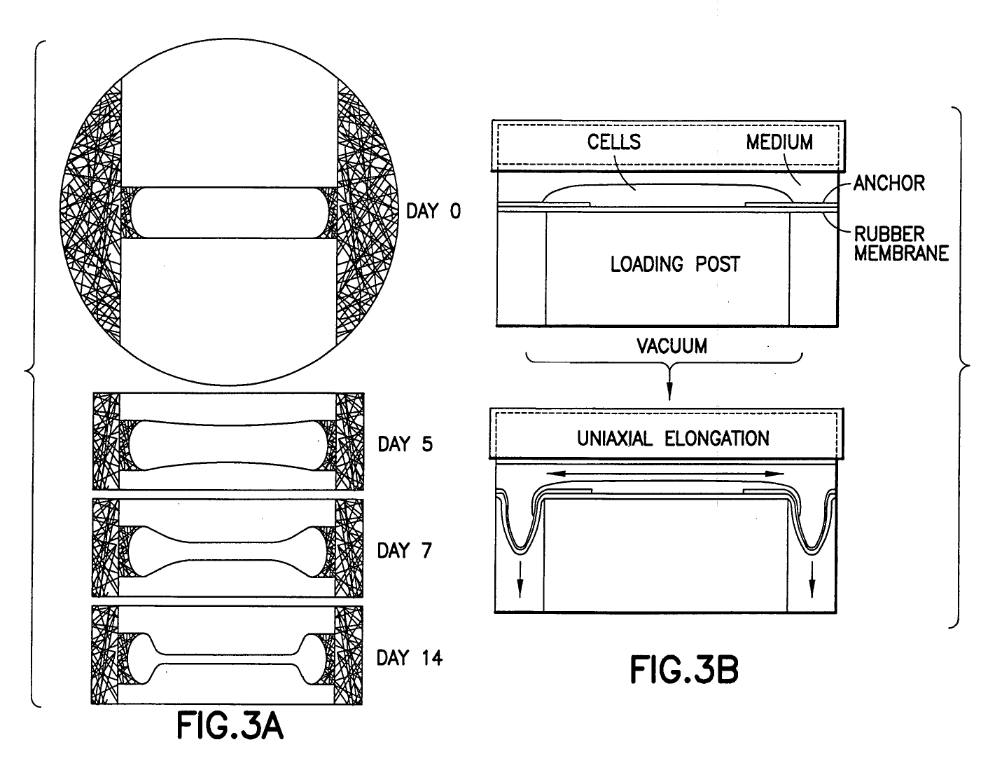

The construct assumed an elongated cylindrical shape, differentiating it from a traditional 2D monolayer culture (FIG. 3A). After 24 h in culture the matrix and cell attachments to the anchor points were mechanically bonded and secured.

Mechanical Loading

BATs™ were uniaxially loaded by placing ARCTANGLE™ loading posts (rectangle with curved short ends) beneath each well of the TissueTrain® plates in a gasketed baseplate and applying vacuum to deform the flexible membranes downward at east and west poles (FIG. 1B; FIG. 3B). The flexible but inelastic non-woven nylon mesh anchors deformed downwards along the long sides of the ARCTANGLE™ loading posts thus applying uniaxial strain along the long axis of each BAT™. The loading regime was 1 h per day at 1% elongation and 1 Hz using a Flexercell® Strain Unit to control the regimen.

Growth Curves

Cell numbers in replicate 2D cultures (n=3/group) were determined every 24 h. Three-dimensional BATs™ were removed from culture with forceps, placed into 15 ml conical tubes containing 1.5 ml of 0.1% collagenase each and incubated at 37° C., 5% CO₂ (n=6 group). Cells were sedimented, resuspended in an equal volume of PBS and cell numbers (n=3/group) determined using a Nub auer hemocytometer.

BAT™ Contraction Index

BATs™ were cultured for up to 8 days. The overall reduction in construct area and volume (defined as remodeling or matrix contraction) as well as the width of the narrowest horizontal region of each BAT™ were determined every 24 h (n=6). Each plate of BATs™ was imaged using a Hewlett-Packard scanner at 600 dpi resolution. Images were analyzed using IMAQ VISION software by National Instruments (Austin, Tex.). The periphery of each BAT™ was outlined to determine the overall area. Each BAT™ was then outlined again to determine the width of the narrowest horizontal region, and a measurement calculated. The width of each BAT™ was measured three times and averaged.

Histology

Three-dimensional BAT™ preparations were fixed in situ with 3.7% paraformaldehyde for 30 min at 25° C. in wells of a TissueTrain® culture plate. After fixation the BATs™ were placed in OTC embedding medium and frozen at −20° C. BATs™ were sectioned into 5 μm thick sections using a cryostat and applied to a glass microscope slide. Sections were stained with hematoxylin and eosin(HTE). Sections were observed and imaged at 10× and 40× magnification using an Olympus BH61 light microscope.

Actin and Nuclear Staining

The BATs™ were fixed, while attached to the anchor points, with 3.7% paraformaldehyde at 25° C. for 30 min. (three BATs™/group). After removal of the fixative, 0.2% Triton X-100 and 0.5% bovine serum albumin (BSA) were added to the BATs™ at 25° C. for 30 min. The solutions were aspirated and the BATs™ were washed three times with PBS. Cells were stained at room temperature (RT) for 1 h with rhodamine phalloidin (200 U/mL, dissolved in methanol) (Molecular Probes 1:400 dilution) to stain polymerized actin and 1 μg/ml of 4′,6-diamidino-2-phenylindin, dihydrochloride (DAPI) (Sigma) to stain nuclei (17, 18). Fluorochromes were diluted in 0.2% Triton X-100 and 0.5% BSA. After 1 h, the fluids were discarded and the constructs were washed three times with PBS. Cells were imaged at 40× magnification using an Olympus BH61 microscope with a 40× objective lens and AnalySIS 3.0 (Soft Imaging System GmbH, Munster, Germany).

Gene Expression Profile of 2D Cultures, 3D Constructs and Native Tendon

Comparative gene expression profiles for cells grown in 2D monolayer cultures, 3D BATs™ and native whole tendon were created using a quantitative reverse transcriptase polymerase chain reaction (RT-PCR) (n=3/group). The experiment was repeated twice. On day 8 of culture, total RNA was isolated from each population using the Qiagen Mini Kit System (Valencia, Calif.). RNA was isolated from whole avian tendon using phenol-chloroform-isoamyl alcohol (PCI) extraction and ethanol precipitation (19). The optical density (OD) of each sample was determined using a Beckman DU640B spectrophotometer to determine the total RNA concentration and purity. RNA samples having an OD from 1.9 to 2.1 were used.

Reverse Transcriptase and Quantitative Polymerase Chain Reaction

The reverse transcriptase reaction was conducted using 1.1 μg of total RNA for each sample (n=3/group) (InVitrogen, Inc.). Each reaction tube was subjected to the following conditions: 25° C. for 10 min; 42° C. for 2 h; 99° C. for 5 min and 5° C. for 5 min (Table 1). Primers were designed using GeneFisher software and synthesized by MWG Biotech (High Point, N.C.). Table 1 includes the primer sequences and PCR product length for each gene. cDNAs were separated in 1.5% agarose gels and identities confirmed by sequence analysis. Expression levels for Collagen I, Collagen III, Collagen XII, decorin, tenascin, fibronectin, prolyl hydroxylase and β-actin were quantitated.

Material Properties of BAT™ Constructs

Engineering stress strain curves were generated for the bioartificial tendon constructs (BATs™) at 7 and 14 days. Tensile tests were performed using an ElectroForce 3200™ (ELF™) mechanical tester by EnduraTEC Systems Corp. (Minnetonka, Minn.), equipped with soft-foam covered micro tissue grips. The modulus of elasticity for each BAT™ was determined by measuring the slope of the linear portion of the engineering stress-strain curve. Ultimate tensile strength was determined by finding the peak stress from this curve.

Each BAT™ subjected to a tensile test was removed from its TissueTrain® anchor point with metal forceps and placed in the center of the grips with approximately one-third of the material secured at each end. Each BAT™ was loaded in tension for a total of 5 mm displacement. All BATs™ failed at less than 5 mm elongation.

The BATs™ initial cross-sectional area (Aₒ) was required to calculate engineering stress (σₑ). This was obtained through the detection of the minimum cross-sectional area, along the length of the BAT™, prior to test initiation (time=0). A custom Labview (National Instruments, Austin, Tex.) program was used to obtain diameter data from two cameras focused on the front and the side, 90° to the front view of the BAT™. The following formulas were used in the program to calculate the engineering stress strain curve.

$${A}_{o} = {[ \frac{π}{4} ( {D}_{camera\_ 1} * {D}_{camera\_ 2} ) ]}_{0 , min}$$

$$Engineering stress ( {σ}_{e} )$$

$${σ}_{e} = \frac{{F}_{t}}{{A}_{o}}$$

where Fₜ=Force at time, t, Aₒ=initial cross-sectional area

$$Engineering Strain ( {ε}_{e} )$$

$${ε}_{e} = \frac{( {y}_{displacement} )}{{L}_{0}}$$

where y_{displacement}=the displacement of the cross-head at time, t

Lₒ=the original length of the BAT™

3. Results

Growth Curve

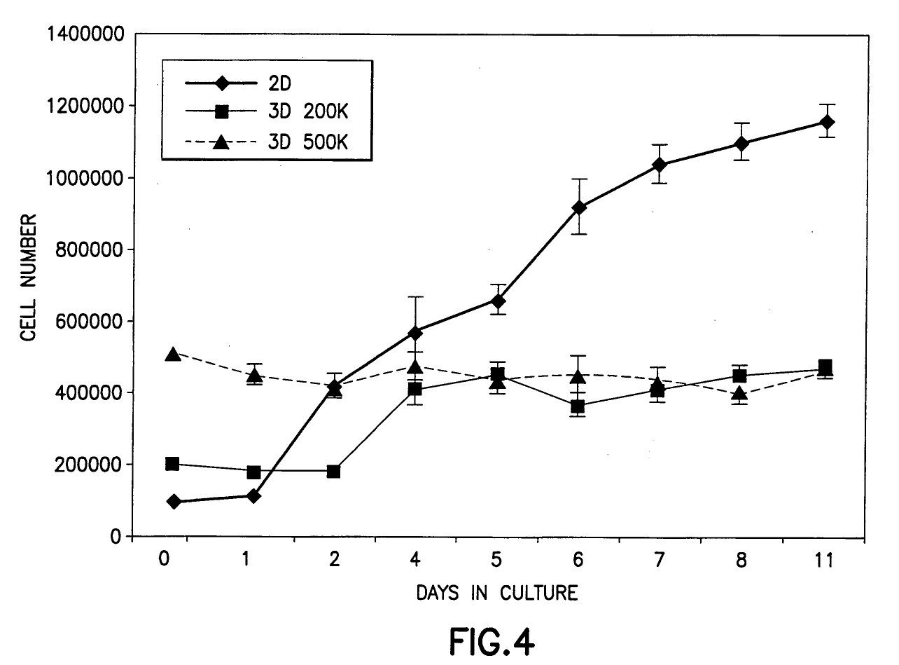

Cells were cultured for up to 11 days. Analyses of ATIFs grown in BATs™ with an initial seeding density of 200,000 cells, and of cells grown in 2D monolayers demonstrated typical lag, log and stationary phases of a traditional growth curve (n=3/group/time period). Both culture conditions also reflected similar generation times: 2D=33 h; 3D 200,000 cells=31 h. However, BATs™ with an initial seeding density of 500,000 cells did not demonstrate a typical log phase, but rather remained in a stationary phase (FIG. 4). Both 3D cultures contained the same number of cells after 11 days. These data indicated that a comparable cell-to-matrix ratio was maintained although the initial seeding densities differed.

Contraction Index

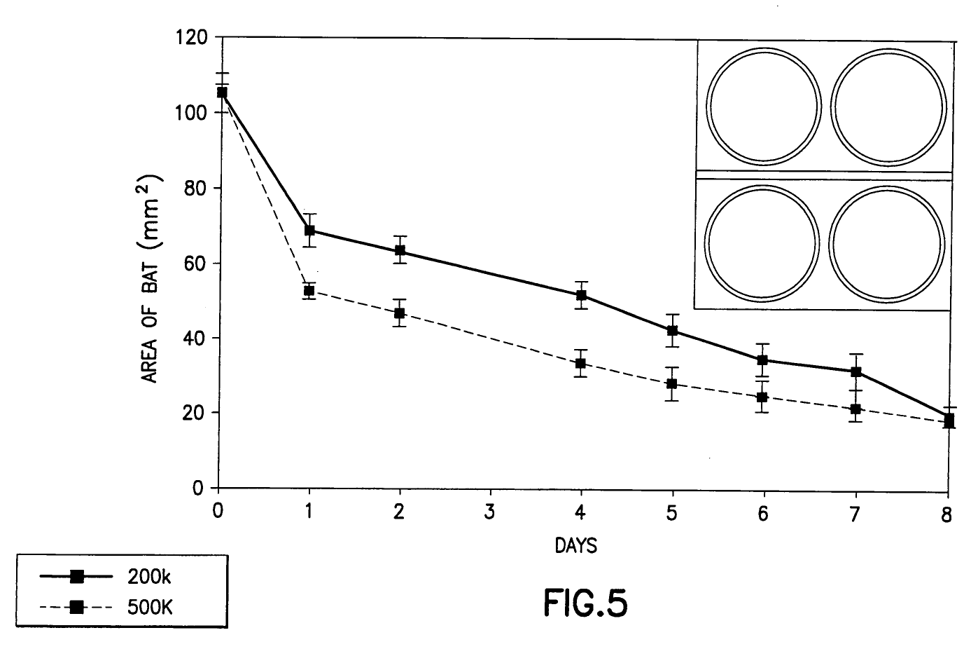

ATIFs in a linear collagen gel attached to matrix-bonded anchor ends to form a 3D “tendinous” construct (n=6/Group). The BATs™ were cultured for up to 11 days and initially assumed a rectangular to cylindrical shape (FIG. 5, inset). As the cells reorganized the collagen matrix, macroscopic radial contraction of the construct was evident. Over an 8 day period, image analysis revealed that the ATIFs contracted the overall area of the construct by 82% (mean+/−SD (p<0.001)), with a reduction in midsection width by 89% (p<0.001) (FIG. 5). Contraction parameters were compared using a one-way ANOVA and least square means post-hoc multiple comparisons (a=0.05).

Histology

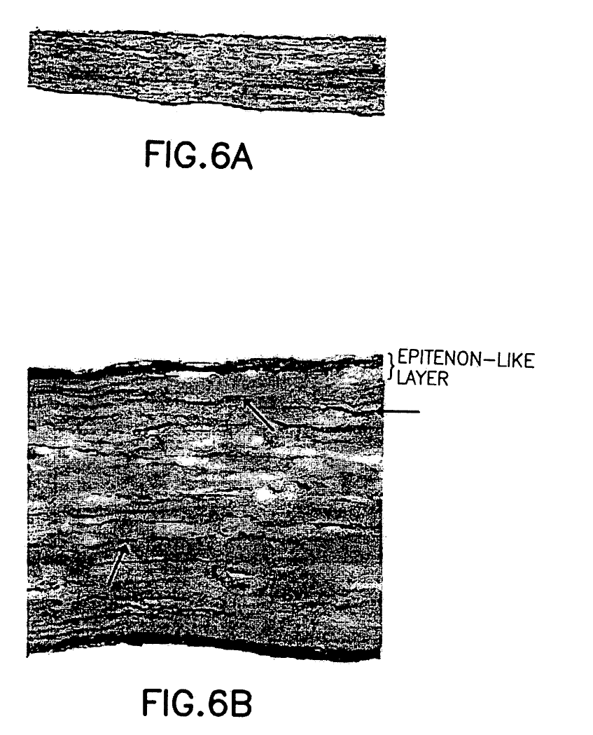

BATs™ stained with hematoxylin and eosin appeared tendon-like demonstrating a multicellular top layer resembling an epitenon and deeper cells aligned in the direction of the long axis of the BAT™ (FIG. 6). Mechanically loaded BATs™ had similarly aligned cells with even more elongate nuclei and cytoplasmic extensions. As with whole tendon, cells were spread and stacked throughout the collagenous matrix. An epitendinous sheath surrounds native whole tendon. This is observed by the more intense hematoxylin nuclear staining of the surface cells. This epitendinous staining is also observed as a dense, basophilic stain in the bioartificial tendons. Together, these data indicated that the appearance of the bioartificial tendon mimicked the histologic appearance of whole native tendon.

Cytoskeletal and Nuclear Staining

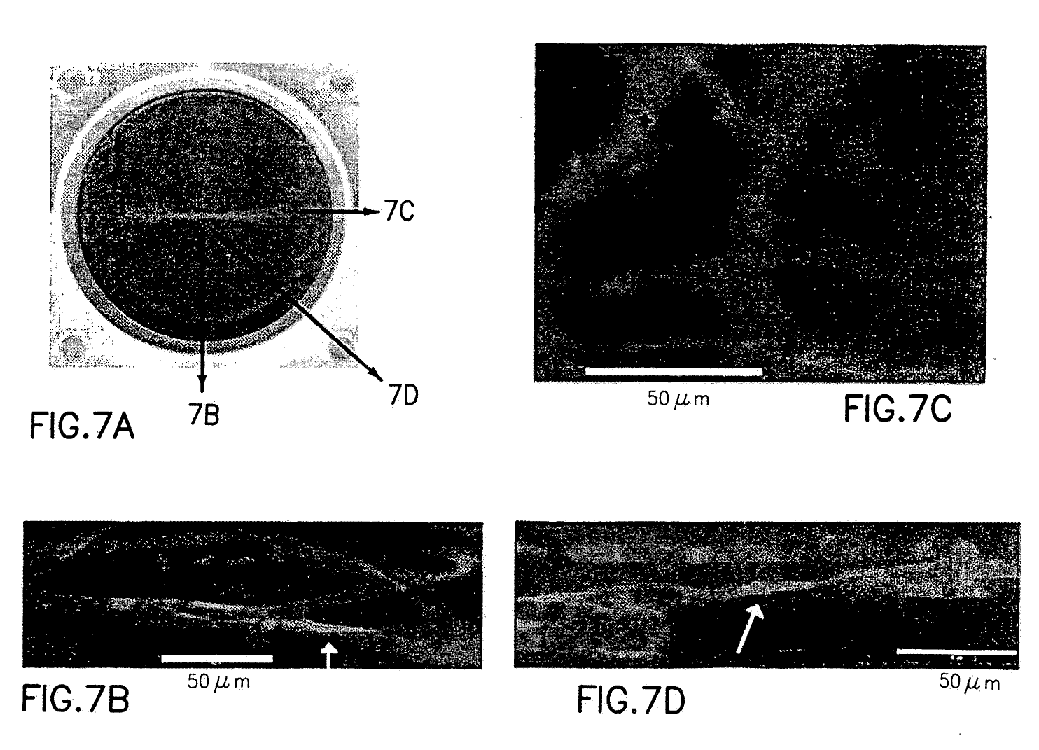

Staining with rhodamine phalloidin (for filimentous actin) and DAPI (for nuclei) showed a three-dimensional view of the cellular architecture of the bioartificial tendons. The cells were elongated and stacked throughout the matrix, similar to the appearance of the hematoxylin and eosin (H&E) stained BATs™. Moreover, numerous cell-to-cell contacts were observed. Cells residing in the midsection of the construct were aligned parallel to neighboring cells. Cells residing toward the end points of the BATs™ were spread in a more random fashion (FIG. 7). This effect occurs due to an increase of intrinsic strain in the central portion of the BAT™. This region of the BAT™ had a smaller cross-sectional area compared to that at the end attachment points. At the initial time of plating, the cells in BATs™ were rounded and demonstrated minimal attachment to the surrounding matrix. Cell spreading increased as time in culture increased. Cells stained at the time of initial plating until approximately day 2 showed minimal polymerized actin cytoskeletons. By day 7 the cell processes were fully extended and formed attachment points to the collagen matrix and surrounding cells. Furthermore, by day 7 in culture, the cells contracted the collagenous matrix substantially. By day 14, gross macroscopic radial contraction was evident. Moreover, microscopically, the cells assembled into a more tendon-like anatomic appearance. The midsection of the BATs™ contained TIFs that were well spread throughout the matrix. The periphery of the BAT™ contained a more organized aggregation of TIFs that resembled an epitenon.

Gene Expression Profile

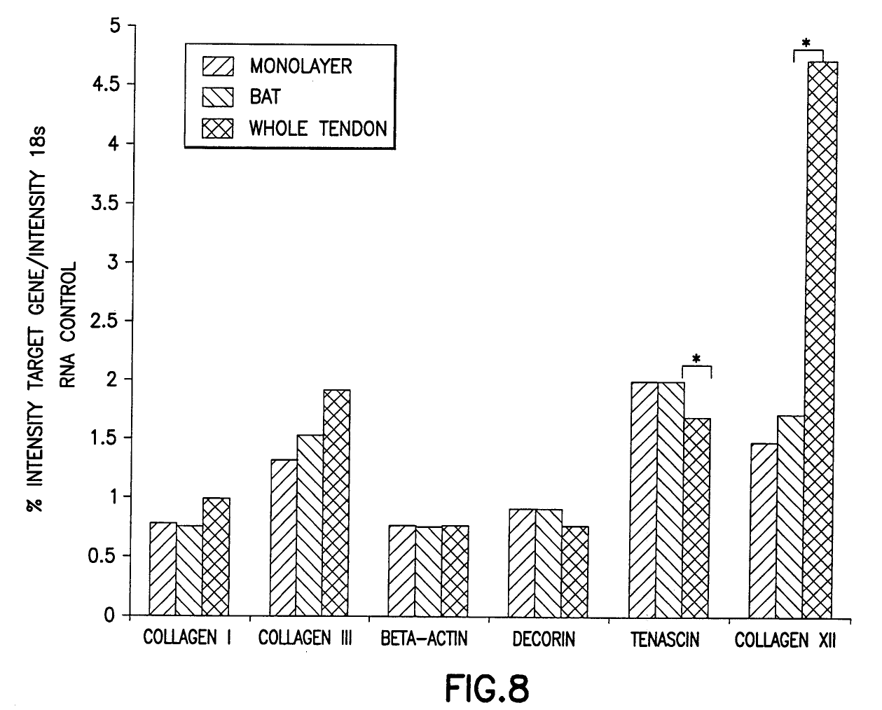

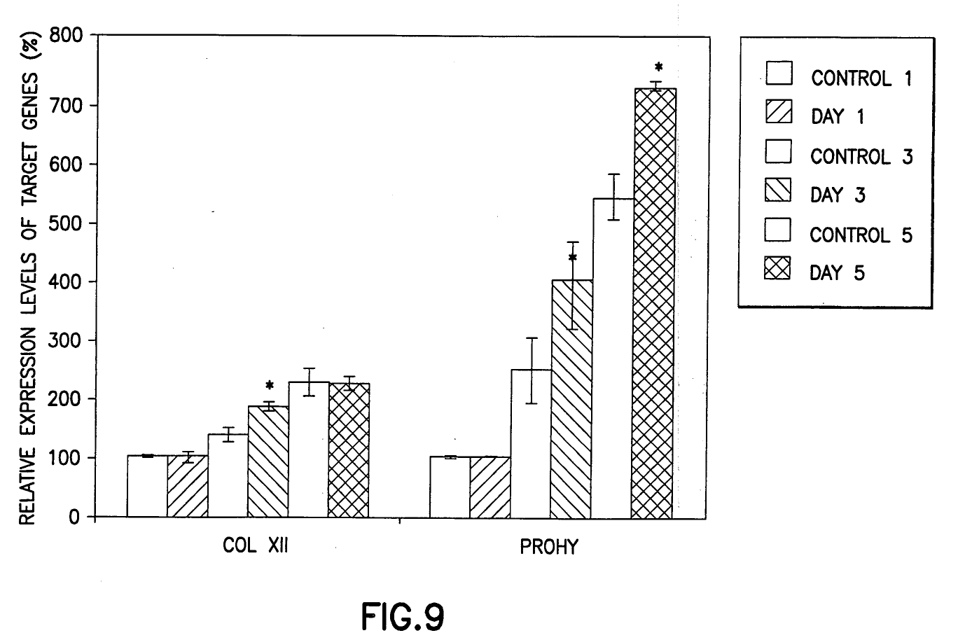

Results of gene expression analyses indicated that all genes tested for were expressed in BATs™ as well as in whole tendon and 2D monolayer cultures (FIG. 8, n=3/group; experiment repeated twice). These data indicated that the ATIFs cultured in the 3D collagenous matrix retained their phenotypic expression profiles for the predominant substrate also retained the genetic expression of the predominant collagens found in tendon cells and did not vary from the expression levels in BATs™. Some explanations for this include a low passage number (p3) and that the 2D tissue culture plate growth surface was treated with Collagen I. The means of these samples passed a Student's t-test and showed no statistically significant difference (p<0.05); α=0.05). The only statistically significant difference in values between samples isolated from whole tendon and those isolated from BATs™ was for genes coding for Collagen XII (60% greater expression in whole tendon) and tenascin (10% less expression in whole tendon) (p<0.05). Mechanical loading increased the mRNA levels of Collagen XII at day 3 by 33% (p<0.05) (FIG. 9). The mRNA level of prolyhydroxylase were increased at day 3 by 61% and by 33% on day 5 (p<0.05).

Mechanical Properties

The modulus of elasticity for control and mechanically loaded BATs™ composed of Collagen 1 and 200,000 chick TIFs was determined on days 7 and 14. At initial plating (day 0), the BATs™ were unable to be subjected to tensile testing due to their weak, gelatinous nature. It was assumed that the modulus at this time point was approximately equal to zero. The modulus of elasticity of the BATs™ increased over time and increased with mechanical conditioning (Table 2). The average modulus for control BATs™ on day 7 was 0.49 MPa, and on day 14 was 0.96 MPa. The average modulus for mechanically conditioned BATs™ on day 7 was 1.8 MPa and on day 14 was 4.3 MPa. The increase in modulus over time may be a direct correlation to the degree of cell attachment and spreading within the collagen matrix. BATs™ subjected to cyclic mechanical load of 1% elongation at 1 Hz for 1 h per day for 7 days had a 2.9 fold greater ultimate tensile strength compared to nonloaded controls (Table 3, p<0.22). At the two week time point, the ultimate tensile strength of nonloaded BATs™ strength increased 6.9 fold compared to the one week value while that of loaded BATs™ increased 2 fold (p<0.36). There was no significant difference in values for ultimate tensile strength between load and no load groups at week two.

4. Discussion

A three-dimensional tenocyte-populated linear bioartificial tendon was created using a novel molding process. The goal was to use a 3D cell culture approach to create a tissue replacement that mimicked the biological behavior and material properties of native tendon. This approach has been explored for creating bioartificial muscle tissue (Kosnik, P. A. et al., Tissue Eng., 7, 573, 2001; Lu, X. et al., Circulation, 104, 594, 2001). It was observed that the tenocytes possessed mitotic ability, functioned to remodel their surrounding matrix and retained their intrinsic phenotypic mRNA expression patterns and appearance. Thus, the hypothesis that tendon internal fibroblasts grown in a tethered, three-dimensional collagenous matrix mimic native tendon in appearance and genetic expression was validated.

The tenocytes dispersed in a collagen gel remodeled and contracted their matrix by an 82% reduction in area over an eight-day period. This confirms what has previously been reported in other systems: that matrix contraction by fibroblasts is typically rapid in the first week of culture (Bellows, C. G. et al., J. Cell Sci., 58, 386, 1981). In vitro cell-populated matrix cultures that are fabricated by combining cells, matrix components and nutrients or other growth factors have been previously reported (see, for example, Bell, E. et al., PNAS, USA, 76, 1274, 1979; Butler, D. et al., J. Cell Physiol., 116, 159, 1983). Fibroblasts incorporated into a collagen gel remodel their matrix in a process that simulates a wound repair sequence. It has been proposed that developmental matrix remodeling may be regulated through cell attachment to the collagen and other matrix molecules (Harris, A. K. et al., Nature, 290, 249, 1981; Stopak, D. et al., Dev. Biol., 90, 383, 1981). During this remodeling process, fibroblasts remodel the collagen matrix to form a uniaxially oriented material in response to the appropriate orientation cues, such as mechanical stress or magnetic fields. The alignment of fibroblasts throughout the BATs™ supports the hypothesis that forces exerted by cells alter the surrounding collagen matrix. This gradual alignment, in turn, can provide the mechanical cues to neighboring cells to orient in a similar pattern.

The immobilized end-point anchors for the BATs™ created the mechanical stresses necessary to develop a uniaxially oriented material with the histology resembling a tendon. As the fibroblasts exerted traction on the collagen matrix, the matrix was consolidated in the unconstrained portions of the culture. Moreover, the collagenous matrix increased in alignment and stiffness along the axis between the two anchored endpoints. The increasing stiffness in the BATs™ may have been the signal for the cells to orient in a direction parallel to the principal strain. It can also be assumed that the intrinsic strain at the central two-thirds of the construct was greater since the construct assumed an hourglass-shaped appearance at that location

$$( σ = \frac{F}{A} ) .$$

There was a 7% greater reduction of the cross-sectional area in this central region when compared to the end-point regions.

Tenocytes in the BATs™ were mitotic; which is consistent with other reports of fibroblasts in three-dimensional collagen matrices (32, 6). However, this is the first report of a growth curve comparing tenocytes grown in two dimensions (monolayer) versus those grown in three dimensions (BATs™). The cells grown in a monolayer and those grown in BATs™ share similar generation times. However, one difference between the two groups was that the cells grown in three-dimensional culture entered into the stationary phase of the growth curve at day 5, while the cells grown in a monolayer continued in the exponential phase of the growth curve.

The mitotic halt may be a result of contact inhibition with neighboring cells. Staining cells in BATs™ with rhodamine phalloidin at the same time point (day 5) showed an overlap between adjacent cells. This probable cellular junction was an indication that intracellular communication may have been established, allowing for transmission of the mechanical signals to exit the cell cycle. Cellular communication occurs through gap junctions. This hypothesis could further be investigated by immunohistochemical staining with anti-connexin-43 antibody, the protein involved in forming the gap junction in both human and avian tenocytes.

A profile of gene expression for some of the principle genes expressed by tenocytes was created. This approach evaluated the RNA expression profile of tenocytes in BATs™ compared to that expressed by cells maintained in a monolayer culture in whole tendon. This evaluation was performed to ensure that the tenocytes grown in the 3D BATs™ retained their genotypic expression patterns.

The expression patterns of genes coding for Collagen I, Collagen III, β-actin and decorin were the same when comparing the RNA isolated from cells in BATs™ to cells in either a 2D monolayer or whole tendon. Expression patterns of the genes coding for tenascin, fibronectin and Collagen XII were the same when compared to cells grown in either monolayer or 3D BAT™ cultures. There was a statistically significant difference between expression profiles for RNA isolated from whole tendon and from BATs™ for genes coding for Collagen XII and tenascin. However, loading increased expression of Collagen XII and prolylhydroxylase. Increased hydroxylase activity could be responsible for greater stability in the collagen fibrils and hence greater ultimate tensile strength. These findings were based on BATs™ that were maintained in culture for 7 days. Lysyl oxidase expression did not change, suggesting that aldehyde creation from epsilon amino groups of lysine or hydroxylysine and subsequent formation of Schiff base crosslinks was unlikely the cause of increased matrix strength (data not shown). It would be worthy of investigation to determine if time in culture would yield a less significant difference between the expression of tenascin and Collagen XII in BATs™. Tenascin is an extracellular matrix protein that is highly expressed during organogenesis and active turnover of the ECM. This may be a plausible reason why the expression of this message was greater in the developing BATs™ than in the adult whole tendon. Collagen XII is a protein that is known to associate with fibrillar collagens. It is speculated that its role is to enhance the binding of cells, proteoglycans or other extracellular matrix proteins to the fibrillar collagen network.

Young's modulus was determined for mechanically conditioned and for control BATs™ at day 7 and 14. Conditioning the BATs™ drove their moduli towards that of mesenchymal stem cells seeded onto a collagen matrix (31.7 MPa). Moduli for various native whole tendons have been reported to average 1.5 GPa for in vitro testing (Bennett, M. B. et al., J. Zool., 209A, 537, 1986) and 1.2 GPa at maximum forces in vivo (Constantinos, M. N. et al., J. Physiol., 521, 307, 1999). The elastic moduli of the BATS™ were significantly lower than native tendon, but a trend of strengthening over time was demonstrated. A qualitative but significant increase in stiffness and decrease in elasticity was observed for each BAT™ over the two-week testing period. It can be hypothesized that a quantitative increase in stiffness would occur over time and could approach a modulus of elasticity close to that of whole tendon.

The biomechanical strength and moduli of the BATs™ was increased by applying cyclic mechanical strain in vitro (Table 2). Moreover, it is believed that an anabolic steroid, nandrolone, in conjunction with cyclic load, can increase strength of BATs™ populated with human supraspinatus tenocytes. Tendons are in a continuous state of dynamic remodeling. Soft musculoskeletal tissues adapt to their mechanical environment by atrophying and weakening in response to immobilization, and strengthening in response to exercise. Application of daily, cyclic mechanical strain can enhance the biomechanical properties of bioartificial tendons.

5. Conclusion

This is the first report describing the fabrication and characterization of a bioartificial tendon using native tendon cells suspended in a Collagen I matrix that can be readily subjected to regulated, cyclic, mechanical loading. Furthermore, this is the first study to characterize a tissue engineered tendon construct histologically, genetically and biomechanically. Tendon internal fibroblasts grown in a tethered, three-dimensional collagenous matrix mimic native tendon in appearance and genetic expression but are weaker in biomechanical strength.

### EXAMPLE 2

### Elasticity of Human Tenocyte-Populated Bioartificial Tendons (BATs™) Increased with IL-1

1. Introduction

In order to find a better therapeutic method for tendon/ligament repair and/or replacement, several in vitro models for engineered tendon have been developed recently (Awad et al., J. Biomed. Mater Res., 51(2), 233-240, 2000). One of them is a BioArtificial Tendon (BAT™) model system utilizing tenocyte-populated molded collagen gels (Awad et al., J. Biomed. Mater Res., 51(2), 233-240, 2000). This 3D BAT™ system allows the testing of tenocyte responses to drugs, cytokines and mechanical loading but is too weak to replace conventional grafts materials. In an attempt to modulate the material properties of the cell-gel composite, the influence of IL-1β on the elasticity of human tendon internal fibroblast (HTIF)-populated bioartificial tendons (BATs™) was investigated. IL-1β has been reported to increase the expression of matrix metalloproteinases (MMPs) and elastin. It was hypothesized that IL-1 might increase the elasticity of BATs™ by up-regulating the expression of elastin and down-regulating matrix protein (Collagen type I) expression. Gene expression was quantified with quantitative RT-PCR. The elasticity of BATs™ was determined by length recovery after stretch. The influence of IL-1β on the actin cytoskeleton and integrin attachment to matrix in BATs™ also was tested +/−cytochalasin D or GRGDTP, respectively.

2. Methods

Primary human tendon internal fibroblasts (HTIFs) were isolated from discarded human tendon tissue as described previously (Banes et al., J. Ortho Res., 6, 73-82, 1988). HTIFs from passage 2 to 4 were used in this study. BATs™ were fabricated at a cell density of 2 million cells/ml collagen gel suspension (Vitrogen). Cells were incubated at 37° C. for 24 h before addition of 100 pM IL-1β and inhibitors. BAT™ images were recorded with a scanner and automated imaging software, ScanFlex™ (Flexcell International Corp.). Medium was refreshed every 24 h. On day 5, BATs™ were collected, total RNA extracted with an RNeasy mini kit (QIAGen), cDNA synthesized with SuperScriptII (Invitrogen) and quantitative PCR carried out using a quantitative PCR kit from Ambion. The PCR products were separated on 2% agarose gels and the bands were quantitated in Photoshop. The elasticity of BATs™ was tested on day 5. BATs™ were subjected to a maximum stretch (20% elongation, 1 Hz for 1 h) and the BAT™ images were recorded for 24 h after stretch.

3. Results

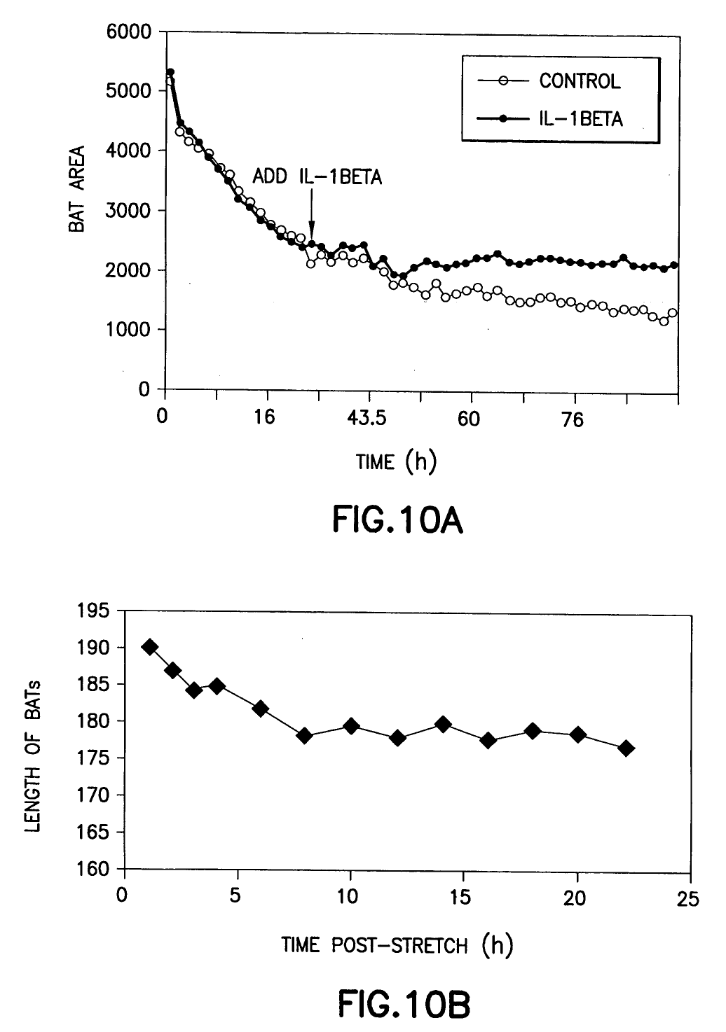

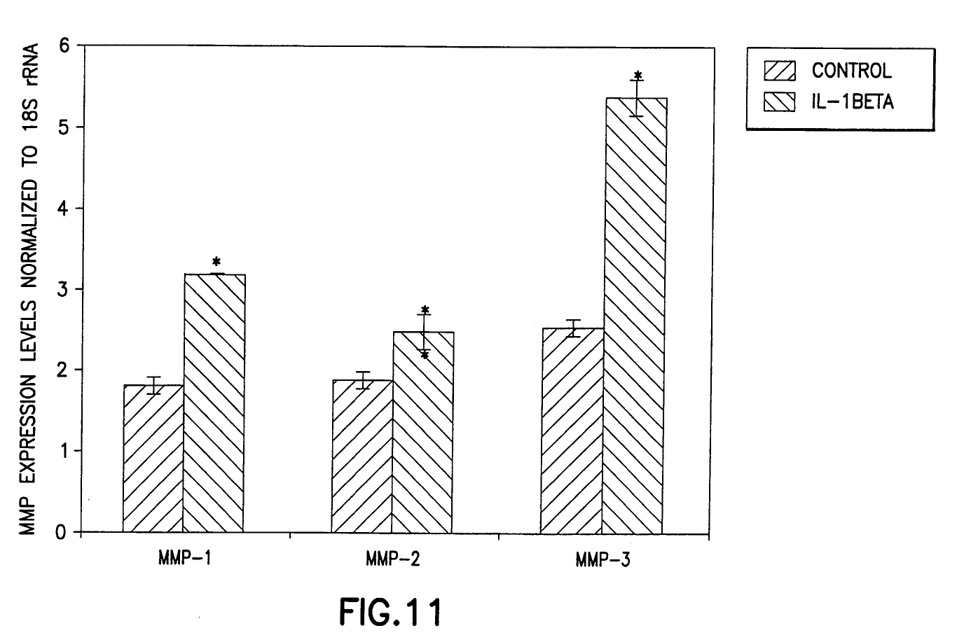

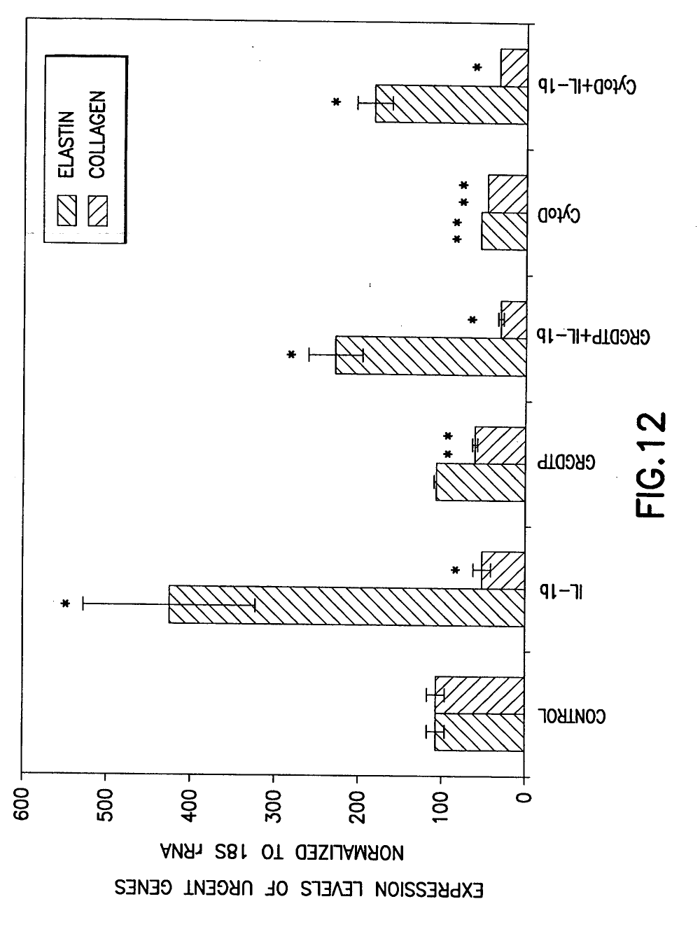

IL-1 reduced the contraction of BATs™ 24 h post addition and increased the elasticity of BATs™ (FIG. 10). IL-1β-treated BATs™ survived the maximum stretch and the elongated BATs™ recovered to original length 8 h post stretch. Gene expression analysis showed that IL-1β up-regulated the expression of MMPs 1, 2, 3 (FIG. 11) and elastin, but down-regulated Collagen type I (FIG. 12). The results with the presence of 10 μM cytochalasin D or 100 μg/ml GRGDTP indicate that blocking integrin attachment to matrix with GRGDTP did not affect elastin mRNA level, but reduced its stimulation by IL-1, indicating that release of some integrin contacts (collagen, fibronectin) and cell shape change without actin depolymerization can affect IL-1β signaling.

4. Discussion

IL-1β has been reported to increase the expression of MMPs and elastin in isolated cells. However, this is the first report that IL-1β increased the elasticity of 3D bioartificial tendons (BATs™). The results indicate that the elasticity of engineered tendon (or other tissues) may be controlled by regulating the expression of collagen and elastin. Although, the mechanism of IL-1β regulation of BAT™ elasticity is not known, it is a mechanism by which the mechanical properties of engineered tendon may be regulated.

### EXAMPLE 3

### IL-1β Reduction of the Modulus of Human Tendon Internal Fibroblasts

1. Introduction

It has been reported that IL-1β can regulate the elasticity of human tendon internal fibroblast (HTIF) populated bioartificial tendons (BATs™) by down-regulating Collagen type I expression and up-regulating elastin expression (Qi, J. et al., ORS, San Francisco, Calif., 2004). The measurement of material properties showed that IL-1β reduced the modulus of BATs™. To address the mechanism, the effects of IL-1β on the expression levels of Collagen type I and elastin at both message and protein levels were investigated. The results showed that IL-1β decreased the expression of Collagen type I, but increased elastin expression. Both extracellular matrix protein and cells contribute to the mechanical properties of BATs™, and it was reported that cytochalasin D decreased cell modulus by up to three fold (Wu, H. W. et al., Scanning, 20, 389-397, 1998). Therefore, it was hypothesized that IL-1β would reduce cell modulus by decreasing the expression of β-actin or disrupting the structure of cytoskeleton. This study investigated the influence of IL-1β on cell modulus and cytoskeleton in human tenocytes.

2. Methods

Primary HTIFs were isolated after surgery from discarded human tendon tissue as described previously (Banes et al., J. Ortho Res., 6, 73-82, 1988). HTIFs from passage 2 to 4 were used in this study. HTIFs were allowed to attach and spread for 24 h before addition of 100 pM IL-1β. Medium was refreshed every 24 h. On day 5, cells were collected for cell modulus measurement and gene expression analysis. Young's modulus of HTIFs was measured by aspirating a cell into the bore of a calibrated micropipette with a calibrated vacuum source. The cell-aspiration process was videotaped for subsequent data analysis to calculate the pipette bore size, the steady state pressure required to aspirate a segment of a cell into the pipette bore and the time constant for aspiration. Cytoskeleton change was monitored by rhodamine-phalloidin staining. The expression levels of β-actin was determined by quantitative RT-PCR. Total RNA was extracted with an RNeasy mini kit (QIAGen), cDNA was synthesized with SuperScriptII (Invitrogen) and quantitative PCR was carried out using 18S rRNA as an internal control (Ambion). The PCR products were separated on 2% agarose gels and pixel intensity of the bands was quantitated in Photoshop.

3. Results

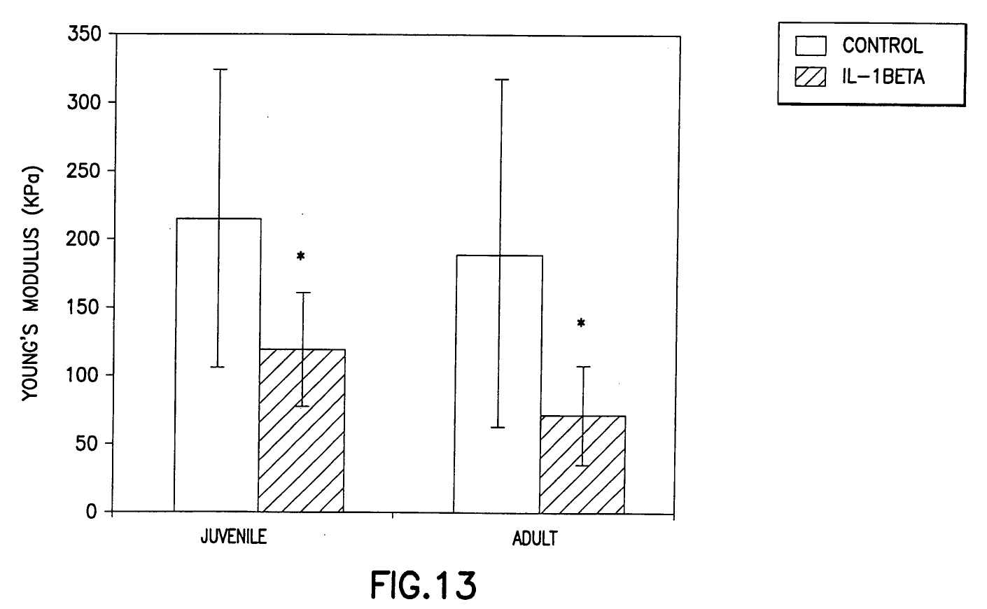

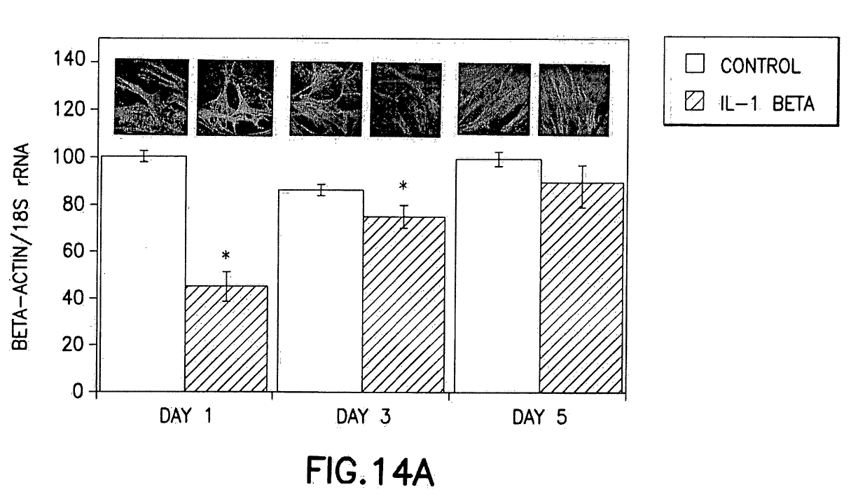

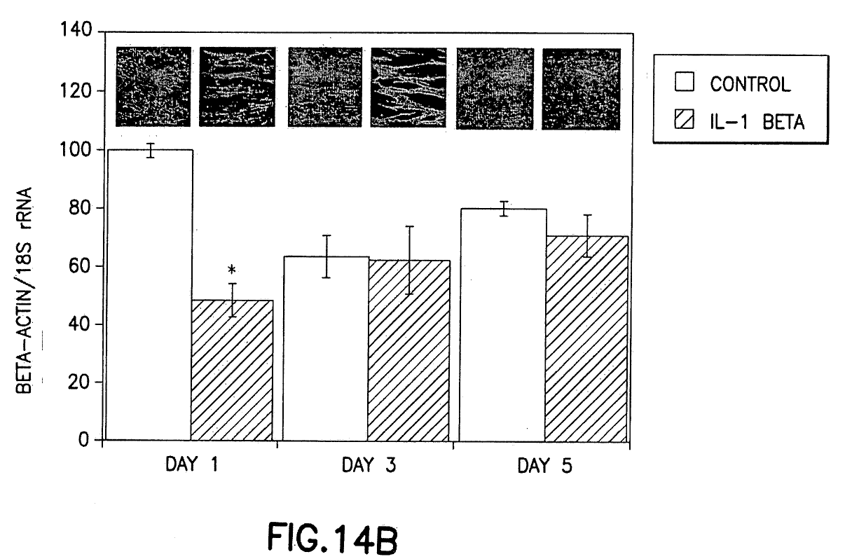

The modulus of HTIFs from two patients, 2 years old and 46 years old, were measured. Fifteen cells from each group were measured. As expected, IL-1β reduced the cell modulus by 45% and 62%, respectively (FIG. 13). Quantitative RT-PCR results for cultured cells showed different time courses for β-actin expression in 2D and 3D BATs™ (FIG. 14). At 24 h post addition of IL-1β, the message of β-actin was reduced by more than 50% in both 2D and 3D cultures, then the message level of β-actin returned to normal at day 3 in 3D cultures. The results of rhodamine-phalloidin staining showed that the protein level of β-actin was also down regulated, but recovered more slowly compared to message recovery (FIG. 14). In 2D cultures, the cytoskeletal structure in about 20% cells was disrupted by IL-1β; the protein level was further reduced at day 3 but mostly recovered at day 5. However, the interrupted cytoskeletal structure was still seen in some cells. The results from 3D bioartificial tendons showed a different time course. The message level of β-actin returned earlier compared to that for 2D cultures, but the protein level recovered more slowly. Even at day 5, the IL-1β treated cells showed much lower fluorescence intensity of rhodamine-phalloidin staining compared to that of control. At days 1 and 3, the intercellular space at the perpendicular direction of BATs™ (north-south direction in the pictures) was increased by more than 100%.

4. Discussion

The results indicated that IL-1: reduced cell modulus by decreasing/disrupting the cytoskeleton. Previous studies indicated that there may be a threshold of intrinsic strain that cells maintain in their mechanical environment. This intrinsic strain modulates the regulation of collagen and elastin expression by IL-1β in HTIFs. The cytoskeletal network plays a critical role in mechano-transduction and strain setpoint in cells. By disrupting the cytoskeleton structure, IL-1β reduced the intrinsic strain in the cells. The results in this study further support the idea that there is a threshold sensor in soft connective tissue cells similar to that for osteoblasts. Under the control of this mechanical sensor, IL-1β was able to regulate extracellular and intracellular strain, preventing cells from dying in an extreme mechanical environment. At day 1, the expression level of β-actin was dramatically reduced when cells were under normal intra- and extracellular strain. Twenty-four hours later, when the extra- and intracellular strain was reduced due to the digestion of matrix proteins and disruption of cytoskeleton, the reduced expression of β-actin by IL-1β was recovering so that the cells could establish a new, but lower, strain setpoint. In future studies, the cell moduli at different time points will be measured to investigate the relationship of cell modulus and cytoskeleton reorganization. It is believed that the reorganized cytoskeleton in the presence of IL-1β resulted in less stiff cells even after the recovery of β-actin expression.

It will be apparent to those skilled in the art that various modifications and variations can be made in the methods of the present invention without departing from the spirit or scope of the invention. Thus, it is intended that the present invention include modifications and variations that are within the scope of the appended claims and their equivalents.

The invention claimed is:

1. A method for manipulating intrinsic strain of cells, comprising treating the cells either in vivo or in vitro with a compound that affects intrinsic strain setpoint of the cells in order to modulate cell-cell connections, cell-matrix connections, extracellular matrix synthesis, secretion, stiffness, organization and/or remodeling, material properties, or attachment of the cells to the matrix via integrins or other integrin-like cell-matrix attachments.

2. The method according to claim 1, wherein the cells comprise an in situ native tissue.

3. The method according to claim 1, wherein the cells comprise an in vitro fabricated tissue engineered construct.

4. The method according to claim 3, wherein the tissue engineered construct is a bioartificial tissue tissue (BAT™) selected from the group consisting of human tendon internal fibroblast (HTIF)-populated tendons, ligaments, menisci, intervertebral discs, cartilage, muscle, fascia and other like connective tissue cells.

5. The method according to claim 4, wherein the BAT™ is populated by autologous or allogeneic cells or stem cells from adult or embryonic sources.

6. The method according to claim 4, wherein the compound is added at the beginning, during or at the end of fabrication of the tissue engineered construct.

7. The method according to claim 1, further comprising applying a mechanical external strain to the cells.

8. The method according to claim 7, wherein the mechanical external strain is comprised of biaxially loading a tissue engineered construct by placing a circular Loading Post™ as a planar faced cylindrical post beneath a well of a culture plate and applying a vacuum to deform a flexible membrane downward so as to apply an equibiaxial strain to a tissue engineered construct.

9. The method according to claim 7, wherein the mechanical external strain is comprised of a combination of a uniaxial and a biaxial mechanical loading of a tissue engineered construct by placing an ARCTANGULAR™ loading post as a rectangle with curved short ends and then placing a circular LOADING POST™ as planar faced cylindrical posts beneath a well of a culture plate, and applying a vacuum to deform a flexible membrane downward so as to apply a uniaxial strain then an equibiaxial strain to a tissue engineered construct.

10. The method according to claim 9, wherein the mechanical external strain is comprised of deformations selected from the group consisting of tension, compression, shear, shear stress by fluid flow and a combination of these deformations, in order to achieve the mechanical loading.

11. The method according to claim 1, wherein the compound is a mediator which causes release or engagement of cell attachment points of the cells from its extracellular matrix.

12. The method according to claim 11, wherein the mediator is selected from the group consisting of binding site peptides, such as collagen, elastin, fibronectin or laminin-binding site peptides; decorin; biglycan; fibromodulin and lumican.

13. The method according to claim 1, wherein the compound is a ligand that modulates attachment and tensional structuring of the cells to the extracellular matrix so as to cause a relaxation or contraction of the cells.

14. The method according to claim 13, wherein the ligand is selected from the group consisting of adenosine triphosphate, adenosine diphosphate, adenosine monophosphate, uridine triphosphate, uridine diphosphate, uridine monophosphate, uridine triphosphate and nonmetabolyzable analogs of these or other like compounds.

15. The method according to claim 13, wherein the ligand is a channel blocker selected from the group consisting of suramin, verapamil, nifedipine and gadolinium.

16. The method according to claim 13, wherein the ligand can be used singly or in combination with other ligands and wherein the ligand or ligands can be used in timed doses.

17. The method according to claim 1, wherein the compound reduces, increases or alters extracellular matrix remodeling.

18. The method according to claim 17, wherein the compound is hyaluronic acid.

19. The method according to claim 1, wherein the compound is a cytokine which adjusts the intrinsic strain of cells by modulating gene expression, said gene expression comprised of cytoskeletal genes that express cytoskeletal proteins selected from the group consisting of actin, myosin, α-actinin, vimentin, vinculin, titin and other binding partner proteins, and genes that express proteins selected from the group consisting of Collagen type I, elastin and matrix metalloproteinase.

20. The method according to claim 19, wherein the cytokine is selected from the group consisting of interleukin-1beta, tumor necrosis factor-alpha, tumor necrosis factor-beta, transforming growth factor-beta1, transforming growth factor-beta3 and connective tissue growth factor.

21. The method according to claim 19, wherein the cytokine is interleukin-1beta.

22. The method according to claim 21, wherein interleukin-1beta increases gene expression of elastin and matrix metalloproteinase and decreases the expression of Collagen type I in the tissue-engineered construct.

23. The method according to claim 21, wherein interleukin-1beta increases elasticity of the tissue-engineered construct.

24. The method according to claim 1, wherein the compound interferes with actin polymerization to decrease or alter modulus of the cell and thus decrease or alter the intrinsic strain of the cell, said compound selected from the group consisting of cytochalasin D, cytochalasin B and other compounds that interfere with actin polymerization or depolymerization.

25. The method according to claim 1, wherein the compound disrupts the microtubular network of the cell and thus increases or alters cell modulus.
